(19) United States
(12) Patent Application Publication
(10) Pub. No.: US 2023/0330115 A1
(43) Pub. Date: Oct. 19, 2023

(54) COMPOSITION COMPRISING COMBINATIONS OF CANNABINOID HOMOLOGUES THCB (TETRAHYDROCANNABUTOL) AND THCP (TETRAHYDROCANNABIPHOROL)

(71) Applicant: Bradley Douglass, Black Diamond, WA (US)

(72) Inventors: Simon Robinson, Seattle, WA (US); Bradley Douglass, Black Diamond, WA (US)

(73) Assignee: Green Sky Creations, LLC, Seattle, WA (US)

(21) Appl. No.: 18/301,206

(22) Filed: Apr. 14, 2023

Related U.S. Application Data
(60) Provisional application No. 63/331,610, filed on Apr. 15, 2022.

(51) Int. Cl. A61K 31/00

(52) U.S. Cl. CPC A61K 31/658

(57) ABSTRACT

The present disclosure provides a cannabinoid composition comprising combinations of at least two THC homologues, wherein the first THC homologue is THCB and the second THC homologue is THCP.

### FIELD OF THE DISCLOSURE

The present disclosure is generally related to compositions including two or more tetrahydrocannabinol (THC) homologues that have superior therapeutic and wellness profiles than THC or other singular THC homologues. The present disclosure more specifically relates to compositions including tetrahydrocannabiphorol (THCP) and tetrahydrocannabutol (THCB).

### SUMMARY OF THE DISCLOSURE

Determining the correct dosage for cannabinoids can be difficult. It is common for users to take too large of a dose of a single cannabinoid, resulting in unintended side-effects (paranoia, anxiety, lethargy, etc.), or to take too small of a dose, leading to inadequate effects (e.g. pain persists). This difficulty in dose-finding is exacerbated by the therapeutic dogma that seeks to find single compounds to achieve the ideal pharmacological effect, i.e. the “silver bullet” approach. In many instances, such as with cannabinoids, this type of pharmacological reductionism makes successful therapeutic outcomes less likely and can increase the incidence of adverse events. Combinations of cannabinoids (i.e. at least two) can offer broader utility in unlocking therapeutic benefit by exhibiting more-nuanced, on-target effects and ameliorating activity that can lead to side effects.

(−)-trans-Δ9-tetrahydrocannabiphorol (THCP, tetrahydrocannabinol-C7) is a potent CB1 and CB2 agonist that was recently isolated in trace amounts from certain cultivars of Cannabis sativa. THCP is structurally similar to Δ9-THC (THC), the main active component of cannabis, and is therefore considered a “THC homologue.” The only structural difference is that the pentyl (5-carbon linear) side chain of THC has been extended to heptyl (7-carbon linear) in THCP. The structure of THC compared to THCP is shown below.

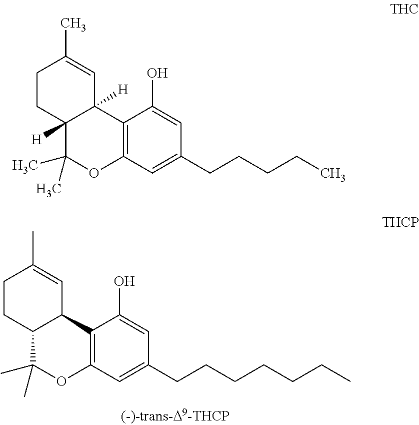

Structure-activity relationship (SAR) investigations have demonstrated that a resorcinol alkyl side-chain length longer than THC (i.e. five carbons) leads to more potent agonist or partial agonist activity at the CB1 receptor. As can be seen in Table 1, the CB1 binding affinity of THCP (1.2 nM) is significantly greater than that of delta-9 THC (18 nM). Furthermore, the effects of THCP in mice include hypomotility, analgesia, catalepsy and decreased rectal temperature, similar to those of THC. These four criteria form the basis of the Tetrad Test that is a means of assessing THC-like pharmacological activity in animals, which are unable to report psychotropic effects using language like humans can.

The binding affinity at CB2 receptors of THCP (6.2 nM) is also significantly greater than THC (42 nM), which is important because substances that have significant CB2 binding can sometimes also exhibit the ability to counteract CB1 activity. For substances like cannabidiol (CBD) this is believed to result from direct competitive inhibition at the CB1 receptor binding pocket, but there is also suggestion that this inhibitory effect partially occurs further downstream at the endocannabinoid system circuit level. This type of negative feedback could stem the effects of the CB1 activity and become self-limiting at certain concentrations.

Due to its potency at low doses, THCP may be useful in addressing certain ailments and conditions for which Δ9-THC may currently be inadequate or ineffective. However, the increased “potency” of THCP necessitates therapeutic formulation options that minimize potential side effects, counter off-target effects, enhance on-target effects, and even potentially rescue individuals administered too large of a dose.

Δ9-Tetrahydrocannabutol (THCB, tetrahydrocannabinol-C4), is another homologue of THC. Where THC possesses a pentyl side-chain (5-carbon linear), THCB possesses a butyl side-chain (4-carbon linear). THCB shows an affinity for the human CB1 (Ki=15 nM) and CB2 receptors (Ki=51 nM) comparable to that of THC. Conducting the formalin test on rodents administered THCB reveals possible analgesic and anti-inflammatory properties. Moreover, the Tetrad Test in mice corroborates CB1 partial agonistic activity. The structure of THCB is shown below.

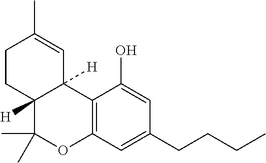 [SMILES: CCCCc1cc(O)c2c(c1)OC(C)(C)[C@@H]1CCC(C)=C[C@@H]21]

THCP experiments in mice, such as the Tetrad Test, elicit responses indicating significantly increased potency (about 30 times greater than THC) at CB1 even at low doses (5 mg/kg of body weight). However, many of the effects do not appear to increase as the dose increases. Thus, the effect of THCP effectively “caps out” to its maximum effect at low doses, and additional doses will not necessarily increase the effect. Conversely THCB exhibits a biphasic effect. At a lower dose THCB has a first effect (phase 1) and at a higher dose, THCB has a different effect (phase 2). In the first phase (10 mg/kg) THCB shows no significant catalepsy, analgesia, or hypolocomotion. Said another way, THCB fails the Tetrad Test at low concentrations. The first phase also includes a small hypothermic effect (less than 0.5° C. decrease). In the second phase (20 mg/kg), THCB exerts a significant direct effect on catalepsy and analgesia, and a slight direct effect on hypolocomotion. This is tantamount to a partial success in the Tetrad Test, which is pharmacologically uncommon. Notably, the hypothermic effect from the first phase appears to reverse in the second phase.

Thus compositions that combine THCB and THCP can be useful in several medical and recreational environments. Such a composition may have limited abuse potential, as the maximum CBx-mediated effect of THCP can be achieved at low doses and doses of greater magnitude may not increase the effect. Moreover, additional dosing with THCB may modulate the overall pharmacology of THCP, and/or partially reverse the hypothermic effects of THCP. It is therefore desirable to create compositions that include THCP and THCB in specific ratios and concentrations to achieve different combinatorial effects. Preferred embodiments of this principle are disclosed in FIG. 2. For example, composition 1 references a dosage form comprised of 1 mg of THCP and 10 mg THCB, or more generally a 1:10 ratio of THCP to THCB. Composition 1 represents a low dose of both THCP and THCB and as such THCP would contribute CB1 activity and positive Tetrad Test activity, but THCB would show negative Tetrad Test activity and a slight hypothermic effect. This type of composition could be useful in treating pain associated with sunstroke or sunburn leading to moderate pain relief and some core temperature modulation. In contrast, composition 3 comprised of 1 mg of THCP and 100 mg THCB represents a low dose of THCP but a high dose of THCB. This type of composition could be useful in treating pain associated with frostbite coincident with exposure where moderate pain relief is demanded, but the reverse hypothermic effect of high-dose THCB is also desirable. Composition 5 comprised of 100 mg of THCP and 1 mg of THCB represents a high dose of THCP and a low dose of THCB. This composition would enable treatment of severe pain, but would minimize the incapacitation or anxiety that occurs in some individuals and results from high dose THC or THCB, alone or in combination, by benefitting from the ceiling on psychoactive effects exhibited by THCP.

There exists a need to reverse the effects of cannabinoids if too high a dose is taken, and furthermore there exists a need to easily cap the effect of cannabinoids, since accidentally administering high doses can have severe and negative effects. Furthermore, there is a need to design therapeutic combinations of known cannabinoids to achieve a formulaic pharmacological effect that is superior to the effects of individual cannabinoids in terms of efficacy, tolerability, and side-effect profile. Properties such as binding affinities for cannabinoid receptors, such as those for various cannabinoids in Table 1, as well as for other receptors are useful for tailoring the activity of these designed combinations:

TABLE 1

| | | Binding Affinity (nM) | | Functional Activity (nM) | |
|---|---|---|---|---|---|
| Cannabinoid Name | Acronym/Abbrev. | CB1 | CB2 | CB1 | CB2 |
| (—)trans-delta-9-tetrahydrocannabinol | Delta-9, THC | 18 ± 4 | 42 ± 9 | 269 ± 36 | 327 ± 43 |
| (—)trans-delta-8-tetrahydrocannabinol | Delta-8 | 78 ± 5 | 12 ± 2 | 5,820 ± 782 | 524 ± 70 |
| (—)trans-delta-7-tetrahydrocannabinol | Delta-7 | ? | ? | ? | ? |
| (—)trans-delta-10-tetrahydrocannabinol | Delta-10 | ? | ? | ? | ? |
| (—)trans-delta-9-tetrahydrocannabinolic acid | THCA | 1,292 ± 89 | 1,650 ± 163 | >10,000 | >10,000 |
| 11-OH-delta-9-tetrahydrocannabinol | 11-Hydroxy | <200 | ? | <200 | ? |
| Cannabidiol | CBD | 2210 ± 558 | 2860 | 1,469 ± 197 | 104 ± 14 |
| Cannabidibutol | CBDB | ? | ? | ? | ? |
| Cannabidiphorol | CBDP | ? | ? | ? | ? |
| Cannabidivarin | CBDV | 503 ± 58 | 3,970 ± 976 | >10,000 | 3 ± 08 |
| Cannabinol | CBN | 368 ± 121 | 168 ± 32 | 307 ± 39 | 289 ± 38 |
| Cannabigerol | CBG | 440 | 337 | >10,000 | 1,158 ± 221 |
| Cannabigerolic acid | CBGA | 4,526 ± 953 | >10,000 | 182 ± 32 | 118 ± 27 |
| Cannabichromanone D | CBCN D | 7,117 ± 1,090 | 2,828 ± 569 | 8 ± 0.9 | 3,945 ± 1,106 |
| (—)trans-delta-9-tetrahydrocannabivarin | THCV | 22 ± 5 | 105 ± 21 | >10,000 | >10,000 |
| (—)trans-delta-9-tetrahydrocannabibutol | THCB | 15 | 51 | ? | ? |
| (—)trans-delta-9-tetrahydrocannabiphorol | THCP | 1.2 | 6.2 | ? | ? |

### BRIEF DESCRIPTIONS OF THE FIGURES

FIG. 1: Illustrates a composition comprising combinations of cannabinoid homologues THCB (tetrahydrocannabutol) and THCP (tetrahydrocannaphorol), according to an embodiment.

FIG. 2: Illustrates a Composition Database, according to an embodiment.

FIG. 3: Illustrates a Dosage Module, according to an embodiment.

FIG. 4: Illustrates a User Device Interface Module, according to an embodiment.

FIG. 5: Illustrates a User Database, according to an embodiment.

### DETAILED DESCRIPTION

The present disclosure details the utility of combinations of tetrahydrocannabinol (THC) homologues, specifically tetrahydrocannabutol (THCB) and tetrahydrocannaphorol (THCP) due to unique and complementary pharmacological effects exhibited by these two compounds, that enables fine-tuning of the pharmacology of cannabinoid mendicants to minimize side effects and tailor effects for specific indications and individuals. Such fine-tuned effects are difficult or impossible to achieve using single cannabinoids, such as THC, and are examples of drug systems intended to interact with complex receptor environments rather than an over-idealized approach of only on-target effects and zero off-target effects. Other cannabinoids and substances found naturally-occurring in cannabis, can be used to further tailor the pharmacological effects of the compositions containing THCP and THCB.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

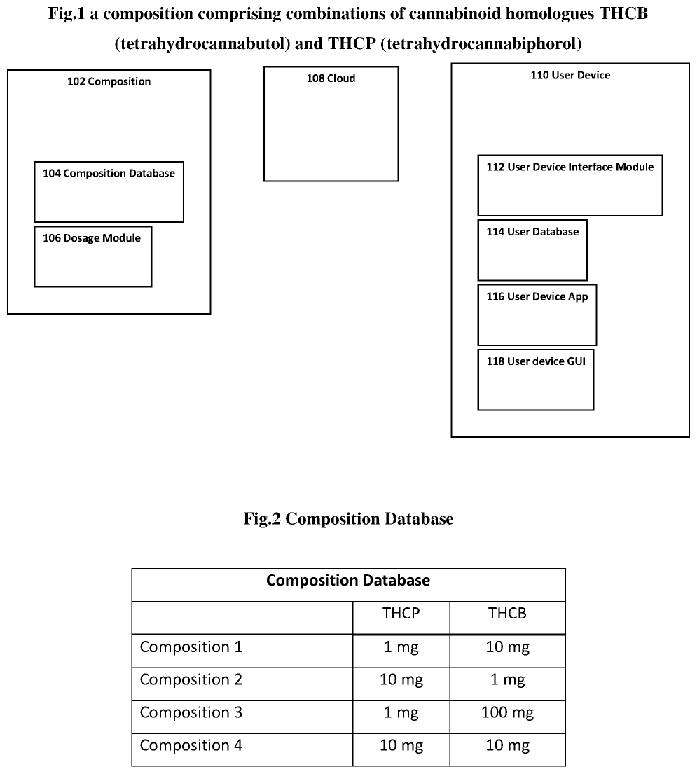

FIG. 1 is a system 100 having a composition 102 for pain treatment of patients comprising combinations of cannabinoid homologues. In one case, the composition 102 may include minor cannabinoids such as, but not limited to, tetrahydrocannabutol (THCB) and tetrahydrocannabiphorol (THCP). Further, the composition 102 may primarily use THCB and THCP instead of delta-9-tetrahydrocannabinol or Δ9-tetrahydrocannabinol (THC) that is the most abundant psychoactive cannabinoid in cannabis. Alternatively, the composition 102 may also include other cannabinoids with a modified sidechain (branched-chain, isobutyl, tert-butyl, etc.) where the modified sidechain occupies smaller or larger steric topography. Additionally, the composition 102 may also include enhancers/time release compositions such as but not limited to, N-acylated fatty amino acid, green tea catechins, piperine, dimethyl sulfoxide (DMSO), and soy lecithin. It can be noted that the composition 102 provides a solution to effects of overdosing and underdosing of the doses since determining the correct dosage for cannabinoids is difficult and it is common to overdose, resulting in unintended side-effects such as, but not limited to, paranoia, anxiety, and lethargy and underdose resulting in no effect and persistence of pain. Since there is no solution to cap the effect of cannabinoids, the overdosing of cannabinoids is common. Further, since there is no expedient way to reverse the effects of THC overdosing, composition 102 provides an important solution to pain management using cannabinoids by combating the effects of overdosing and underdosing. Further, the composition 102 may be used for its effect (or lack of effect) on several dimensions such as, but not limited to, catalepsy, analgesia, hypolocomotion, hypothermia, hypomotility, etc. In some embodiments, the dose may be doubled for differing effects, or a second dose of equal size may be subsequently administered to reverse or modulate the effects of the first dose. Further, the composition 102 may include antidepression including a novel cannabinoid and a botanical extract, the composition 102 may include at least one cannabinoid, e.g., THCB, and at least one botanical extract, e.g., St. John's Wort. In one embodiment, the composition 102 may include other anti-inflammatory agents such as but not limited to, acetaminophen and ibuprofen. It can be noted that the compositions 102 may be in any one or a combination of forms, such as pills, capsules, tablets, transdermal patches, injections, tinctures, smokable herbs, vaporizers, or any other form may be administered orally, topically, intravenously, intranasally, inhaled, taken as a suppository, or some other drug delivery method. Further, the composition 102 includes a cannabinoid formulation comprising a maximum effective dose of THCP with a low amount of other cannabinoids (THC), such that increasing the magnitude of the dose to augment some effects of the formulation can be achieved while maintain a reduced risk of (over) intoxication. Further, the composition 102 provides a cannabinoid product with a maximum effective dose of THC with a low quantity of other cannabinoids (THC) where the increased dosage of the cannabinoid formulation does not significantly increase intoxication. Further, the cannabinoid formulation reduces body temperature of the user and provides an effective dose of THCP and at least one other botanical extract or pharmaceutical composition for reducing the user's body temperature (e.g., fever reduction). Further, the composition 102 includes a biphasic dosing schema for a THCB treatment through the first dose of a composition 102 comprising THCB and at least a second dose of a composition 102 comprising THCB, wherein the first dose of a composition 102 comprising THCB may not produce the effects of THCB, however, the second dose triggers the biphasic effect. Further, the embodiments may include various components of the composition 102, information, properties, and behavior of the components. It should be noted that the molecules with identical structures, but different sidechains are collectively known as homologues and different homologues interact differently with human cannabinoid receptors (CBx i.e., CB1 and CB2) and thus create different effects in the user. Further, making changes to significant pharmacophores of THC and CBD increases, decreases, and modulates the activity at CBx and other receptors such as, but not limited to, transient receptor potential ankyrin 1 (TRPA1). For example, as C-3 chain length increases for THC and THC homologues, CB1 binding affinity increases while the reverse is true for the TRPA1 receptors. While a decrease of C-3 chain length of CBD to three carbons (from five carbons) dramatically increases the binding affinity of CBDV at CB2 receptors. Further, technologies such as, but not limited to, AlphaFold are heralded as “solving” the puzzle of predicting how proteins may fold. It should be noted that the machine learning algorithm of AlphaFold is able to optimize “through-space” energetic interactions of pairs of amino acid residues, rather than simply optimizing single amino acid interactions along the linear peptide chain. Therefore, the THCP, THCB, cannabidiphorol (CBDP), cannabidibutol (CBDB), and other cannabinoids may be used in combinations, to “tune” pharmaceutical effects that are unachievable by individual molecules. Further, a through-space energetics optimization algorithm may be applied to a drug+receptor [binding site] pair, like that used by the AlphaFold, and may help in increasing the reliability and predictability of extrapolating receptor affinity to physiological activity. Further, experiments demonstrate that the dosing effects of THCP significantly increases (about 30 times as potent as THC) effect, even at very low doses. However, many of the effects do not appear to increase as the dose increases. Thus, the effect of THCP effectively “caps out” to its maximum effect at low doses, and additional doses will not necessarily increase the effect. Further, the effect of THC shows the identical effect on THCP at doses of 5 mg/Kg and 10 mg/Kg in hypolocomotion, catalepsy, analgesia, and hypomotility (notably, however, hypothermia does appear to increase at higher doses). Conversely THCB, with a shorter alkyl sidechain, has a biphasic effect. Thus, a lower dose of THCB will have a first effect (phase 1) and a higher dose of THCB will have a different effect (phase 2). Therefore, in the first phase THCB has no significant catalepsy, analgesia, or hypo locomotion and a small hypothermic effect (less than 0.5° C. decrease). In the second phase, THCB has very significant catalepsy and analgesia effect, and a slight hypolocomotion effect and the hypothermic effect from the first phase reversal in the second phase. Consequently, a composition 102 that combines THCB and THCP is useful in several medical and recreational environments. Such a composition 102 has limited adverse event potential, as the maximum effect of the THCP is received at low doses, and additional or increased dosing beyond this does not increase the effect. Moreover, additional dosing of THCB can cause other effects or partially reverse the hypothermic effects of THCP. Therefore, it can be desirable to create a composition 102 that includes THCP and THCB in various ratios to tailor it for the indication and the individual. Such compositions can optionally contain other cannabinoids as well. Further, the relative amounts of THCP and THCB, and other cannabinoids, may be selected algorithmically by modelling the effects based on the affinity for cannabinoid receptors (CB1 and CB2, TRPA1) using machine learning software like the AlphaFold. Further, embodiments may utilize a composition database 104 that includes one or more compositions 102. Further, the embodiments may include a dosage module 106 that may continuously monitor biometric input data from a user. Further, embodiments can optionally include a cloud 108, the cloud or communication network may be a wired and/or a wireless network. The communication network, if wireless, may be implemented using communication techniques such as Visible Light Communication (VLC), Worldwide Interoperability for Microwave Access (WiMAX), Long Term Evolution (LTE), Wireless Local Area Network (WLAN), Infrared (IR) communication, Public Switched Telephone Network (PSTN), Radio waves, and other communication techniques are known in the art. The communication network may allow ubiquitous access to shared pools of configurable system resources and higher-level services that can be rapidly provisioned with minimal management effort, often over the Internet and relies on sharing of resources to achieve coherence and economies of scale, like a public utility, while third-party clouds enable organizations to focus on their core businesses instead of expending resources on computer infrastructure and maintenance. Further, embodiments can optionally include user devices 110 such as a smartwatch, a smart patch, a chest strap, or other body-worn device. Further, the user device 110 may also include a computing device, laptop, smartphone, tablet, computer, smart speaker, or I/O devices. Input devices may include keyboards, mice, trackpads, trackballs, touchpads, touch mice, multi-touch touchpads and touch mice, microphones, multi-array microphones, drawing tablets, CMOS sensors, accelerometers, infrared optical sensors, pressure sensors, magnetometer sensors, angular rate sensors, depth sensors, proximity sensors, ambient light sensors, gyroscopic sensors, or other sensors. Further, the user device 110 may be configured to monitor user biometrics such as but is not limited to, temperature, heart rate, and hypolocomotion. Output devices may include video displays, graphical displays, speakers, headphones, inkjet printers, laser printers, and 3D printers. Devices may include a combination of multiple input or output devices, including, e.g., Touch screens, physical buttons, microphones, fingerprint readers, accelerometers, vibration devices, etc. Some devices allow gesture recognition inputs by combining some of the inputs and outputs. Some devices allow for facial recognition which may be utilized as an input for different purposes including authentication and other commands. devices allow for voice recognition and inputs, including, e.g., Microsoft KINECT, SIRI for iPhone by Apple, Google Now or Google Voice Search. Additional mobile devices have both input and output capabilities, including, e.g., haptic feedback devices, touchscreen displays, or multi-touch displays. Touchscreen, multi-touch displays, touchpads, touch mice, or other touch sensing devices may use different technologies to sense touch, including, e.g., capacitive, surface capacitive, projected capacitive touch (PCT), in-cell capacitive, resistive, infrared, waveguide, dispersive signal touch (DST), in-cell optical, surface acoustic wave (SAW), bending wave touch (BWT), or force-based sensing technologies. Some multi-touch devices may allow two or more contact points with the surface, allowing advanced functionality including, e.g., pinch, spread, rotate, scroll, or other gestures. Some touchscreen devices, including, e.g., Microsoft PIXELSENSE or Multi-Touch Collaboration Wall, may have larger surfaces, such as on a table-top or a wall, and may also interact with other electronic devices. Some I/O devices, display devices or group of devices may be augmented reality devices. The I/O devices may be controlled by an I/O controller. The I/O controller may control one or more I/O devices, such as e.g., a keyboard and a pointing device, e.g., a mouse or optical pen. Furthermore, an I/O device may also allow storage and/or an installation medium for the computing device. In still other embodiments, the computing device may allow USB connections (not shown) to receive handheld USB storage devices. In further embodiments, an I/O device may be a bridge between a system bus and an external communication bus, e.g., a USB bus, a SCSI bus, a FireWire bus, an Ethernet bus, a Gigabit Ethernet bus, a Fiber Channel bus, or a Thunderbolt bus. Further, the user device 110 could be an optional component and would be utilized in a situation in which the paired wearable device is utilizing the user device 110 as additional memory or computing power or connection to the internet. Further, embodiments may include a user device interface module 112 that may be triggered when the user logs-in to a user device app 116. Further, embodiments may utilize the user database 114 that may contain the data received by the user device interface module 112. Further, embodiments may include a user device app 116, which may run on the user device 110. In one case, the user device app 116 is a mobile application. In another case, the user device app 116 is a web application. Further, the user device app 116 may provide a user device GUI 118 to the user for various operations such as, but not limited to, displaying user profile, track the usage of the of composition 102 over time, log the dose taken by the user, track the dose regimen of the user, enabling the user to communicate with their physician or health care provider. Further, embodiments may include a user device Graphical User Interface (GUI) 118 that may either accept inputs from users or facilitates outputs to the users or may perform both the actions. In one case, a user can interact with the interface(s) using one or more user-interactive objects and devices. The user-interactive objects and devices may comprise user input buttons, switches, knobs, levers, keys, trackballs, touchpads, cameras, microphones, motion sensors, heat sensors, inertial sensors, touch sensors, or a combination of the above. Further, the interface(s) may either be implemented as a Command Line Interface (CLI), a GUI, a voice interface, or a web-based user-interface. In one embodiment, the user device GUI 118 may send notifications in a user-friendly or interactive form to the user.

Functioning of the “Composition Database” will now be explained with reference to FIG. 2.

FIG. 2. shows a composition database 104 that includes one or more compositions 102. It can be noted that various compositions 102 may include various drugs in various proportions, but the key novelty is the inclusion of both THCB and THCP. In some embodiments, the compositions can optionally include cannabidiol (CBD), tetrahydrocannabinol (THC), cannabigerol (CBG), cannabichromene (CBC), tetrahydrocannabinolic acid (THCA), cannabidiolic acid (CBDA), cannabinol (CBN), cannabicyclol (CBL), cannabivarin (CBV), tetrahydrocannabiorcol), tetrahydrocannabivarin (THCV), cannabidivarin (CBDV), cannabichromevarin (CBCV), cannabigerovarin (CBGV), cannabigerol monomethyl ether (CBGM), cannabielsoin (CBE), and cannabicitran (CBT). In another embodiment, the compositions 102 can optionally include other components found in cannabis such as but not limited to, terpenes and flavonoids. For example, the composition database 104 includes a composition 1 containing a dosage of 1 mg of THCP and 10 mg of THCB. Further, the composition database 104 may also include amounts required of one or more components of the compositions 102 based on various biometric data such as, but not limited to, body temperature, latency for moving, latency for the first sign of pain, and distance. It can be noted that the body temperature may be associated with fever of the user, the latency for moving with catalepsy of the user, the latency for the first sign of pain with analgesia, and the distance with hypolocomotion. For example, 5 mg/kg of the compound THCP may lower the body temperature of the user by 0.5° C.

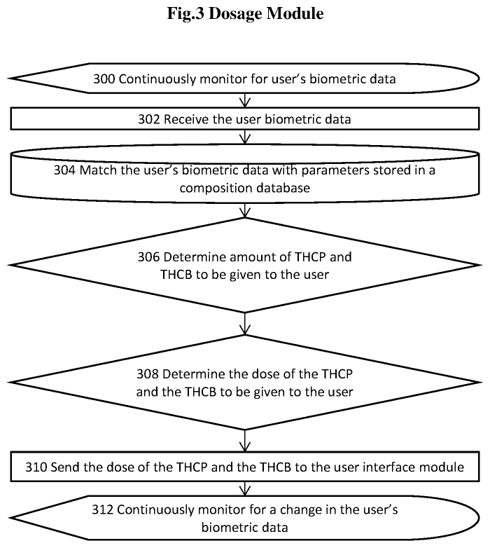

Functioning of the Dosage Module will now be explained with reference to FIG. 3.

FIG. 3 shows the Dosage Module. At first, the dosage module 106 may continuously monitor, at step 300, a user's biometric data. In one embodiment, the user's biometric data may include, but is not limited to a user's, body weight, body temperature, and pain level. Further, the dosage module 106 may receive, at step 302, the user's biometric data from a user device interface module 112. For example, the dosage module 106 receives user Anthony's weight i.e., 110 kgs and body temperature—41° C. from the user device interface module 112 associated with user Anthony. Further, the dosage module 106 may match, at step 304, the user's biometric data with parameters stored in the composition database 104. For example, the dosage module 106 matches user Anthony's body temperature i.e., 41° C. with the parameter such as change in body temperature of −0.5° C. stored in the composition database 104. Based on matching the user's biometric data with the parameters stored in the composition database 104, the dosage module 106 may determine, at step 306, the amount of the THCP and the THCB to be given to the user. For example, the dosage module 106 determines that 1 mg of the THCP and 10 mg of THCB has to be given to user Anthony, based on the matching. Based on the determined amount of the THCP and the THCB at step 306 and the received user biometric data at step 302, the dosage module 106 may determine, at step 308, the dose of the THCP and the THCB. The user may create the determined dosage through a variety of means, for example, by measuring liquid extract using a pipette, by selecting a variety of capsules with incremental dosages (e.g., various capsules in increments of 1 mg and 5 mg of active ingredient), by counting the number of puffs from a extract vaporizer (e.g., each puff from a vaporizer may be a metered dosage of approximately 1 mg of active ingredient), by applying a measured amount of topical application (e.g., a topical ointment for which each teaspoon contains 2 mg of active ingredient), or any other such method. Further, the dosage module 106 may send, at step 310, the dose of the THCP and the THCB to the user device interface module 112. Further, the dosage module 106 may continuously monitor, at step 312, for a change in the user biometric data. In one case, if there is a change in the user biometric data, then the dosage module 106 may return to step 302 to receive the user's biometric data from the user device interface module 112. For example, the dosage module 106 receives a change in user Anthony's body temperature changes from 41° C. to 39° C. In another case, if there is no change in the user biometric data, then the dosage module 106 may end the program. Thereafter, the program ends, at step 314.

Any composition disclosed herein can advantageously include any other pharmaceutically acceptable carriers which include those that do not produce significantly adverse, allergic, or other untoward reactions that outweigh the benefit of administration, such as sesame oil, glycerin or medium-chain triglycerides. Exemplary pharmaceutically acceptable carriers and formulations are disclosed in Remington's Pharmaceutical Sciences, 18th Ed, Mack Printing Company, 1990. Moreover, formulations can be prepared to meet sterility, pyrogenicity, general safety, and purity standards as required by U.S. FDA Office of Biological Standards and/or other relevant foreign regulatory agencies.

For administration, therapeutically effective amounts and ratios (also referred to herein as doses) can be initially estimated based on results from in vitro assays and/or animal model studies. Such information can be used to more accurately determine useful doses and ratios in subjects of interest. The actual dose amount and ratio administered to a particular subject can be determined by a physician, veterinarian or researcher considering parameters such as physical and physiological factors including target, body weight, severity of condition, type of condition, stage of condition, previous or concurrent therapeutic interventions, idiopathy of the subject and route of administration.

Useful doses can range from 0.1 to 5 μg/kg or μg/kg or from 0.5 to 1 μg/kg. In other examples, a dose can include 1 μg/kg, 15 μg/kg, 30 μg/kg, 50 μg/kg, 55 μg/kg, 70 μg/kg, 90 μg/kg, 150 μg/kg, 350 μg/kg, 500 μg/kg, 750 μg/kg, 1000 μg/kg, 0.1 to 5 mg/kg or from 0.5 to 1 mg/kg. In other non-limiting examples, a dose can include 0.1 mg/kg, 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

Useful ratios of THCP to THCB can included 500:1, 100:1, 50:1, 25:1, 20:1, 15:1, 10:1, 8:1, 5:1, 3:1, 2:1, 1:1, 1:2, 1:3, 1:5, 1:8, 1:10, 1:15, 1:20, 1:25, 1:50, 1:100 and 1:500.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly).

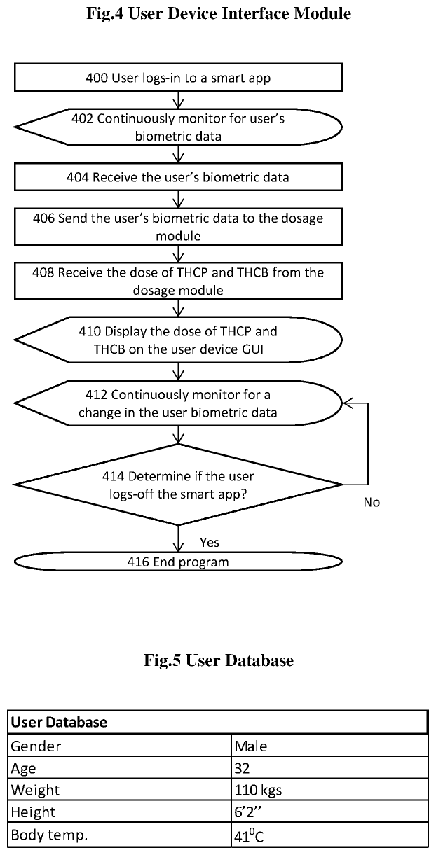

Functioning of the User Device Interface Module will now be explained with reference to FIG. 4.

FIG. 4. shows a user device interface module 112 that may be triggered when the user logs-in to a user device app 116. Further, the user device interface module 112 may facilitate the user to enter the user's biometric data and view the amount of the THCP and the THCB required by the user on a user device GUI 118. Further, the user device interface module 112 may continuously monitor for user's biometric data. In one embodiment, the user device interface module 112 may facilitate the user to input the user's biometric data. It can be noted that the user device interface module 112 may store the user's biometric data in a user database 114. Further, the user device 112 may prompt the user with various question to facilitate the user to input data. For example, the user device interface module 112 may prompt user Anthony with questions such as, but not limited to, “What is the user's gender?”, “What is the user's age?”, “What is the user's weight?”, “What is the user's height?”, and “What is the user's present body temperature?”. In one embodiment, the user device interface module 112 may provide options in one or more question to facilitate the user to choose such as, but not limited to, male and female options for the question “What is user's gender?” and a 34-450 C rating meter for the question “What is the user's present body temperature?”. In another case, the user device interface module 112 may receive the user's biometric data automatically from one or more sensors associated with the user device 110. Further, the user's biometric data may include, but is not limited to, a user's weight, user's body temperature, and user's pain level. Further, the user device interface module 112 may retrieve the user's biometric data, from the user database 114. For example, the user device interface module 112 retrieves user Anthony's weight i.e., 110 Kgs and body temperature, i.e. 41° C. Further, the user device interface module 112 may send the user's biometric data to the dosage module 106. For example, the user device interface module 112 sends the user biometric data i.e., weight i.e., 110 kgs and body temperature—41° C., associated with user Anthony, to the dosage module 106. Further, the user device interface module 112 may receive the dose of the THCP and the THCB from the dosage module 106. In some embodiments, the user device interface module 112 may correlate changes in user biometrics with various doses/ratios of the THCP and the THCB. Further, the user device interface module 112 may display the dose of the THCP and the THCB on the user device GUI 118. For example, the user device interface module 112 displays that user Anthony requires 500 mg of THCP and 1.0 mg of THCB on the user device GUI 118 associated with user Anthony. In one embodiment, the user device interface module 112 may allow the user or caregiver to monitor the effects or side-effects of the composition 102 in real-time and historically. Thereafter, the user device interface module 112 may also constantly monitor if the user logs-off from the user device app 116. In one case, when the user does not log-off from the user device app 116, then the user device interface module 112 may return to continuously monitor for the user's biometric data. In another case, when the user logs-off from the user device app 116, then the user device interface module 112 may end the program. Thereafter, the program ends.

Functioning of the User Database will now be explained with reference to FIG. 5.

FIG. 5 shows a user database 114 that may contain the data received by the user device interface module 112. Further, the user database 114 contains the user admin data such as gender, age, weight, height, and body temperature. For example, the user admin data for user Anthony includes gender—Male, age—32, weight—110 kgs, height—6′2″, and body temperature—41° C. In one embodiment, the user database 114 may facilitate the user to refer to user data for future purposes.

The functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

All of the literature articles cited below should be considered background material and context for this disclosure and are explicitly incorporated by reference.

### GS006—THC AND CBD ANALOGUE COMBOS: BINDING AFFINITY CITATIONS

1. Linciano P, Citti C, Luongo L, Belardo C, Maione S, Vandelli M A, Forni F, Gigli G, Lagana A, Montone C M, Cannazza G. Isolation of a High-Affinity Cannabinoid for the Human CB1 Receptor from a Medicinal Cannabis sativa Variety: Δ⁹-Tetrahydrocannabutol, the Butyl Homologue of 49-Tetrahydrocannabinol. J Nat Prod. 2020 Jan. 24; 83(1):88-98. doi: 10.1021/acs.jnatprod.9b00876. Epub 2019 Dec. 31. PMID: 31891265.
2. Man-Hee Rhee, Zvi Vogel, Jacob Barg, Michael Bayewitch, Rivka Levy, Lumir Hanus̆, Aviva Breuer, and Raphael Mechoulam. Cannabinol Derivatives: Binding to Cannabinoid Receptors and Inhibition of Adenylylcyclase Journal of Medicinal Chemistry 1997 40 (20), 3228-3233 DOI: 10.1021/jm970126f
3. Radwan M M, ElSohly M A, El-Alfy A T, Ahmed S A, Slade D, Husni A S, Manly S P, Wilson L, Seale S, Cutler Si, Ross S A, Isolation and Pharmacological Evaluation of Minor Cannabinoids front High-Potency Cannabis sativa. J Nat Prod. 2015 Jun. 26; 78(6): 271-6. doi: 10.1021/acs.jnatprod.5b00065. Epub 2015 May 22. PMID: 26000707; PMCID: PMC4880513.
4. Husni A S, McCurdy C R, Radwan M M, Ahmed S A, Slade D, Ross S A, ElSohly M A, Cutler S J. Evaluation of Phytocannabinoids from High Potency Cannabis sativa using In Vitro Bioassays to Determine Structure-Activity Relationships for Cannabinoid Receptor 1 and Cannabinoid Receptor 2. Med Chem Res. 2014 Sep. 1; 23(9):4295-4300. doi: 10.1007/s00044-014-0972-6. PMID: 25419092; PMCID: PMC4235762.
5. Pertwee R G. The diverse CB1 and CB2 receptor pharmacology of three plant cannabinoids: delta9-tetrahydrocannabinol, cannabidiol and delta9-tetrahydrocannabivarin. Br J Pharmacol. 2008; 153(2):199-215. doi:10.1038/sj.bjp.0707442
6. McPartland J M. Glass M. Pertwee R G. Meta-analysis of cannabinoid ligand binding affinity and receptor distribution: interspecies differences. Br J Pharmacol. 2007; 152(5); 583-593, doi.:10.1038/sj.bjp.0707399
7. Citti, C., Linciano, P., Russo, E. et al. A novel phytocannabinoid isolated from Cannabis sativa L. with an in vivo cannabimimetic activity higher than Δ⁹-tetrahydrocannabinol: Δ⁹-Tetrahydrocannabiphorol. Sci Rep 9, 20335 (2019). https://doi.org/10.1038/s41598-019-56785-1
8. Petrov R R, Knight L, Chen S R, Wager-Miller J, McDaniel S W, Diaz F, Barth F. Pan H L, Mackie K, Cavasotto C N, Diaz P. Mastering tricyclic ring systems for desirable functional cannabinoid activity. Eur J Med Chem. 2013 November; 69:881-907. doi: 10.1016/j.ejmech.2013.09.038. Epub 2013 Sep. 29. PMID: 24125850; PMCID: PMC3909471.
9. Dutta A K, Ryan W, Thomas B F, Singer M, Compton D R, Martin B R, Razdan R K. Synthesis, pharmacology, and molecular modeling of novel 4-alkyloxy indole derivatives related to carmabimimetic aminoalkyl indoles (AAIs). Bioorg Med Chem. 1997 August; 5(8):1591-600, doi: 10.1016/s0968-0896(97)00111-9. PMID: 9313864.
10. Rinaldi-Carmona M, Barth F, Héaulme M, Shire D, Calandra B, Congy Martinez S, Maruani J. Néliat G, Caput D, et al. SR141716A, a potent and selective antagonist of the brain cannabinoid receptor. FEBS Lett, 1994 Aug. 22; 350(2-3):240-4. doi: 10.101610014-5793(94)00773-x. PMID: 8070571.

What is claimed is:

1. A cannabinoid composition of at least two THC homologues comprising:
(i) tetrahydrocannaphorol (THCP)
(ii) tetrahydrocannabutol (THCB) and,
(iii) wherein the THCP and the THCB combination create a cannabinoid composition capable of a capped and/or biphasic effect.

2. The cannabinoid composition of claim 1, further comprising a pharmaceutically acceptable carrier.

3. The cannabinoid composition of claim 1, formulated for administration by buccal, ingestible, inhaled, intramuscular, intranasal, intravenous, topical, transdermal, and rectal dosage forms.

4. The cannabinoid composition of claim 1, wherein the THCP to THCB ratio within the composition is 1:10, 1:9, 1:8, 1:7, 1:6, 1:5, 1:4, 1:3, 1:2, 1:1, 2:1; 3:1, 4:1, 5:1, 6:1, 7:1, 8:1, 9:1, or 10:1.

5. The cannabinoid composition of claim 1, further comprising terpenoids.

6. The cannabinoid composition of claim 1, further comprising flavonoids.

7. The cannabinoid composition of claim 1, further comprising a cannabinoid selected from cannabidiol (CBD), cannabidibutol (CBDB), cannabidiphorol (CBDP), tetrahydrocannabinol (THC), cannabigerol (CBG), cannabichromene (CBC), tetrahydrocannabinolic acid (THCA), cannabidiolic acid (CBDA), cannabinol (CBN), cannabicyclol (CBL), cannabivarin (CBV), tetrahydrocannabiorcol), tetrahydrocannabivarin (THCV), cannabidivarin (CBDV), cannabichromevarin (CBCV), cannabigerovarin (CBGV), cannabigerol monomethyl ether (CBGM), cannabielsoin (CBE), and cannabicitran (CBT).

8. A kit comprising THCP (Δ-9-THC-C7) and THCB (Δ-9-THC-C4).

9. A method of creating a cannabinoid composition capable of a capped effect and/or a biphasic effect comprising admixing THCP and THCB within a carrier.

10. A method of claim 9, wherein the carrier comprises sesame oil, glycerin or medium-chain triglycerides.

11. A cannabinoid composition of at least two THC homologues comprising:
(i) a first THC homologue
(ii) a second THC homologue, such as tetrahydrocannabutol (THCB) and,
(iii) wherein the first and second THC homologue combination create a cannabinoid composition capable of a capped and/or biphasic effect.
